(19) United States
(12) Patent Application Publication
(10) Pub. No.: US 2026/0042823 A1
(43) Pub. Date: Feb. 12, 2026

(54) SINGLE-CHAIN ECULIZUMAB ANTIBODY-TRANSFERRIN FUSION PROTEIN FOR ENHANCED EFFICACY AND INDICATIONS

(71) Applicant: RNA Therapeutics, Inc., Deerfield, IL (US)

(72) Inventors: Matthias Magoola, Kampala (UG); Sarfaraz K. Niazi, Deerfield, IL (US)

(21) Appl. No.: 18/796,212

(22) Filed: Aug. 6, 2024

(51) Int. Cl. C07K 16/18; C07K 14/79

(52) U.S. Cl. CPC C07K 16/18; C07K 14/79; C07K 2317/24; C07K 2317/622; C07K 2319/50

(57) ABSTRACT

The efficacy and indication of eculizumab do not depend on the Fc region and are subject to transit across cell walls. They can be expanded by using their scFvs conjugated with N-methyl lobe of transferrin protein connected with an environment-sensitive cleavable linker to prevent exocytosis of the scFv yielding high exposure inside body cells such as in the brain, eye, and cancer cells that overexpress transferrin receptors.

### BACKGROUND OF THE INVENTION

Many antibodies do not need their Fc region to bind to target proteins or be efficacious. The binding to target proteins is mediated by the Fab (fragment antigen-binding) region of the antibody, which includes the variable regions that specifically recognize and bind to the target antigen. The Fc region interacts with Fc receptors on immune cells, triggering various immune responses such as antibody-dependent cellular cytotoxicity (ADCC) and antibody-dependent cellular phagocytosis (ADCP). These mechanisms are essential for eliminating target cells, but this action is unnecessary. Many of these antibodies cross cell walls that can be challenging, reducing their efficacy, such as antibodies crossing the blood-brain barrier, the blood-eye barrier and also entering cancerous cells, among many other situations where transcytosis promotion can increase their efficacy, and in some cases find new applications. Table 1 shows antibodies that can function without the Fc region and would benefit from transcytosis assistance, such as binding with transferrin protein or its fragments. The present invention provides a platform for creating a fusion protein that will be substantially more effective than the source antibody.

TABLE 1
Antibodies Effective as scFvs Benefiting from Transcytosis Augmentation

| | | | | | Potential Benefit |
|---|---|---|---|---|---|
| | | Target | Target | Reason for scFv | of Transferrin |
| # | Antibody | Disease | Antigen | Effectiveness | Conjugation |
| 1 | Adalimumab | Autoimmune | TNF-α | Neutralization of | Potential improved |
| | | diseases | | TNF-α to reduce | delivery to |
| | | | | inflammation. | inflamed tissues. |
| 2 | Aducanumab | Alzheimer's | Amyloid- | Aggregated | Increases transit |
| | | Disease | beta | forms of | across the blood- |
| | | | | amyloid-beta | brain barrier and |
| | | | | (plaques) | reduces exocytosis |
| 3 | Aflibercept | Macular | VEGF-A, | Traps VEGF-A, | Enhanced delivery |
| | | degeneration | VEGF-B, | VEGF-B, and | to retinal tissues. |
| | | | PlGF | PlGF to inhibit | |
| | | | | angiogenesis. | |
| 4 | Alemtuzumab | Multiple | CD52 | Directly | Enhanced delivery |
| | | sclerosis, CLL | | targeting CD52- | to lymphocytes in |
| | | | | positive cells for | the brain or other |
| | | | | depletion. | TfR-expressing |
| | | | | | tissues. |
| 5 | Basiliximab | Transplant | IL-2 | Blocking IL-2 | Enhanced |
| | | rejection | receptor | receptor on | targeting of |
| | | | | activated T-cells. | activated T-cells in |
| | | | | | transplant patients. |
| 6 | Belimumab | Systemic | BLyS (B- | Neutralization of | Improved targeting |
| | | lupus | lymphocyte | BLyS to reduce | of B-cells in lupus |
| | | erythematosus | stimulator) | B-cell survival. | patients. |
| 7 | Bevacizumab | Various | VEGF | Direct inhibition | Improved targeting |
| | | cancers, eye | | of VEGF to | and delivery |
| | | diseases | | prevent | across the BBB. |
| | | | | angiogenesis. | |
| 8 | Brolucizumab | Macular | VEGF-A | N/A already a | Enhanced delivery |
| | | degeneration | | scFv | to retinal tissues. |
| 9 | Canakinumab | Periodic | IL-1β | Neutralization of | Improved delivery |
| | | fever | | IL-1β to reduce | to inflamed |
| | | syndromes | | inflammation. | tissues. |
| 10 | Cetuximab | EGFR- | EGFR | Blocking EGFR | Enhanced |
| | | positive | | signaling to | targeting of |
| | | cancers | | inhibit tumor | EGFR-positive |
| | | | | growth. | tumors with high |
| | | | | | TfR expression. |
| 11 | Cinpanemab | Parkinson's | Alpha- | Aggregated | Increases transit |
| | | Disease | synuclein | alpha-synuclein | across the blood- |
| | | | | (Lewy bodies) | brain barrier and |
| | | | | | reduces exocytosis |
| 12 | Crenezumab | Alzheimer's | Amyloid- | Multiple forms | Increases transit |
| | | Disease | beta | of amyloid-beta, | across the blood- |
| | | | | including | brain barrier and |
| | | | | oligomers and | reduces exocytosis |
| | | | | fibrils | |
| 13 | Donanemab | Alzheimer's | Amyloid- | Modified form | Increases transit |
| | | Disease | beta | of amyloid-beta | across the blood- |
| | | | | with | brain barrier and |
| | | | | pyroglutamate at | reduces exocytosis |
| | | | | the N-terminus | |
| | | | | (N3pG) | |
| 14 | Dupilumab | Atopic | IL-4 | Blocking IL-4 | Improved delivery |
| | | dermatitis, | receptor | receptor to | to inflamed |
| | | asthma | | inhibit allergic | tissues. |
| | | | | inflammation. | |
| 15 | Eculizumab | PNH, aHUS | Complement | Inhibition of | Enhanced delivery |
| | | | protein | complement | to complement- |
| | | | C5 | protein C5 to | activated tissues. |
| | | | | prevent | |
| | | | | complement | |
| | | | | activation. | |
| 16 | Emicizumab | Hemophilia | Factor IXa | Mimicking | Potential for |
| | | A | and Factor | factor VIII | improved delivery |
| | | | X | activity to | and function in |
| | | | | promote blood | clotting pathways. |
| | | | | clotting. | |
| 17 | Exidavnemab | Parkinson's | Alpha- | Toxic forms of | Increases transit |
| | | Disease | synuclein | alpha-synuclein | across the blood- |
| | | | | | brain barrier and |
| | | | | | reduces exocytosis |
| 18 | Gantenerumab | Alzheimer's | Amyloid- | Both fibrillar and | Increases transit |
| | | Disease | beta | non-fibrillar | across the blood- |
| | | | | amyloid-beta | brain barrier and |
| | | | | | reduces exocytosis |
| 19 | Gosuranemab | Alzheimer's | Tau | N-terminal tau | Increases transit |
| | | Disease | | fragments | across the blood- |
| | | | | | brain barrier and |
| | | | | | reduces exocytosis |
| 20 | Guselkumab | Psoriasis | IL-23 | Blocking IL-23 | Improved delivery |
| | | | | to reduce | to psoriatic tissues. |
| | | | | inflammation. | |
| 21 | Infliximab | Autoimmune | TNF-α | Neutralization of | Potential improved |
| | | diseases | | TNF-α to reduce | delivery to |
| | | | | inflammation. | inflamed tissues. |
| 22 | Ixekizumab | Psoriasis | IL-17A | Neutralization of | Potential improved |
| | | | | IL-17A to reduce | delivery to |
| | | | | inflammation. | inflamed tissues. |
| 23 | Lecanemab | Alzheimer's | Amyloid- | Soluble amyloid- | Increases transit |
| | | Disease | beta | beta protofibrils | across the blood- |
| | | | | | brain barrier and |
| | | | | | reduces exocytosis |
| 24 | MEDI1341 | Parkinson's | Alpha- | Monomeric and | Increases transit |
| | | Disease | synuclein | aggregated forms | across the blood- |
| | | | | of alpha- | brain barrier and |
| | | | | synuclein | reduces exocytosis |
| 25 | Natalizumab | Multiple | Integrin α4 | Blocking | Enhanced delivery |
| | | sclerosis, | | integrin α4 to | to inflamed tissues |
| | | Crohn's | | prevent immune | or across the BBB. |
| | | | | cell infiltration. | |
| 26 | Ocrelizumab | Multiple | CD20 | Depletion of | Improved targeting |
| | | sclerosis | | CD20-positive | of B-cells in the |
| | | | | B-cells. | brain or other |
| | | | | | tissues. |
| 27 | Ofatumumab | Chronic | CD20 | Depletion of | Enhanced |
| | | lymphocytic | | CD20-positive | targeting of B-cells |
| | | leukemia | | B-cells. | in leukemia |
| | | | | | patients. |
| 28 | Prasinezumab | Parkinson's | Alpha- | Aggregated | Increases transit |
| | | Disease | synuclein | alpha-synuclein | across the blood- |
| | | | | (Lewy bodies) | brain barrier and |
| | | | | | reduces exocytosis |
| 29 | Ranibizumab | Macular | VEGF-A | Direct inhibition | Enhanced delivery |
| | | degeneration | | of VEGF-A to | to retinal tissues. |
| | | | | prevent | |
| | | | | angiogenesis. | |
| 30 | Ravulizumab | PNH, aHUS | Complement | Inhibition of | Enhanced delivery |
| | | | protein | complement | to complement- |
| | | | C5 | protein C5 to | activated tissues. |
| | | | | prevent | |
| | | | | complement | |
| | | | | activation. | |
| 31 | Risankizumab | Psoriasis | IL-23 | Blocking IL-23 | Improved delivery |
| | | | | to reduce | to psoriatic tissues. |
| | | | | inflammation. | |
| 32 | Romiplostim | Immune | Thrombopoietin | Mimicking | Enhanced |
| | | thrombocytopenia | receptor | thrombopoietin | targeting to bone |
| | | | | to increase | marrow. |
| | | | | platelet | |
| | | | | production. | |
| 33 | Romosozumab | Osteoporosis | Sclerostin | Inhibition of | Potential for |
| | | | | sclerostin to | enhanced delivery |
| | | | | promote bone | to bone tissues. |
| | | | | formation. | |
| 34 | Secukinumab | Psoriasis, | IL-17A | Neutralization of | Potential improved |
| | | ankylosing | | IL-17A to reduce | delivery to |
| | | spondylitis | | inflammation. | inflamed tissues. |
| 35 | Semorinemab | Alzheimer's | Tau | Extracellular tau | Increases transit |
| | | Disease | | tangles | across the blood- |
| | | | | | brain barrier and |
| | | | | | reduces exocytosis |
| 36 | Siltuximab | Castleman's | IL-6 | Neutralization of | Improved delivery |
| | | disease | | IL-6 to reduce | to inflamed |
| | | | | inflammation. | tissues. |
| 37 | Solanezumab | Alzheimer's | Amyloid- | Soluble amyloid- | Increases transit |
| | | Disease | beta | beta monomers | across the blood- |
| | | | | | brain barrier and |
| | | | | | reduces exocytosis |
| 38 | Teprotumumab | Thyroid eye | IGF-1 | Blocking IGF-1 | Improved targeting |
| | | disease | receptor | receptor to | of eye tissues in |
| | | | | reduce | thyroid eye |
| | | | | inflammation | disease. |
| | | | | and fibrosis. | |
| 39 | Tilavonemab | Alzheimer's | Tau | N-terminal tau | Increases transit |
| | | Disease | | fragments | across the blood- |
| | | | | | brain barrier and |
| | | | | | reduces exocytosis |
| 40 | Tildrakizumab | Psoriasis | IL-23 | Blocking IL-23 | Improved delivery |
| | | | | to reduce | to psoriatic tissues. |
| | | | | inflammation. | |
| 41 | Tocilizumab | Rheumatoid | IL-6 | Blocking IL-6 | Improved targeting |
| | | arthritis | receptor | receptors to | of inflamed |
| | | | | inhibit | tissues. |
| | | | | inflammatory | |
| | | | | signaling. | |
| 42 | Trastuzumab | HER2- | HER2 | Inhibition of | Enhanced |
| | | positive | | HER2 signaling | targeting of HER2- |
| | | cancers | | pathways. | positive tumors |
| | | | | | with high TfR |
| | | | | | expression. |
| 43 | Ustekinumab | Psoriasis, | IL-12 and | Blocking IL- | Improved delivery |
| | | Crohn's | IL-23 | 12/IL-23 | to inflamed |
| | | disease | | signaling to | tissues. |
| | | | | reduce | |
| | | | | inflammation. | |
| 44 | Zagotenemab | Alzheimer's | Tau | Extracellular tau | Increases transit |
| | | Disease | | tangles | across the blood- |
| | | | | | brain barrier and |
| | | | | | reduces exocytosis |

### BRIEF SUMMARY OF THE INVENTION

Monoclonal antibodies (mAbs) have transformed the treatment modalities for diseases that were until recently assumed untreatable, such as cancer, macular degeneration, and psoriatic arthritis as examples. mAbs have a molecular weight of about 150 kDa and comprise variable light and heavy chains and the constant structure known as Fc region. While the Fc region is not necessary for the initial binding to target proteins, it is crucial for mediating immune effector functions, enhancing therapeutic efficacy, and prolonging the antibody's half-life. A whole antibody's scFv (single-chain variable fragment) is designed to retain the antigen-binding properties, including the variable regions responsible for recognizing and binding to the target antigen without the same effector functions or extended half-life as total antibodies. The smaller size of scFv, around 25-30 kDa, about 15% of the total antibody, allows for easier penetration in tissues and, in many instances, less hindered binding to the target protein. These property attributes are critical to discovering new indications, better safety profiles, and greater manufacturing ease and cost, whether by recombinant process or encoding using mRNA templates.

The design of scFv can be established using a series of bioinformatics and molecular biology steps to recognize and join the domains of a known antibody sequence, particularly an antibody known to have little utility of Fc fragment. Initially, the antibody's nucleotide or amino acid sequence, including variable and constant regions of the heavy and light chains, is obtained. Bioinformatics tools such as IMGT/V-QUEST and IgBlast are used to analyze the sequence, identifying and annotating the V, D, and J gene segments for the heavy chain and the V and J gene segments for the light chain. ANARCI is then utilized to number the antibody sequence according to IMGT, Chothia, or Kabat numbering schemes, helping to pinpoint the exact positions of the variable and constant domains.

The VH domain, located at the N-terminus of the heavy chain, includes the V gene segment with three CDRs interspersed with four FRs, ending where the CH1 domain begins. Similarly, the VL domain at the N-terminus of the light chain consists of the V gene segment, comprising three CDRs and four FRs, ending where the CL domain starts. Following the VH domain in the heavy chain sequence, the CH domains (CH1, CH2, CH3, and CH4 if present) are identified based on known consensus sequences for the constant regions of the antibody isotype. The CL domain follows the VL domain in the light chain sequence and is identified by comparing it to known consensus sequences for the constant region of the light chain.

Once the VH and VL domains are identified, they are joined by aligning their sequences to ensure correct reading frames, confirming that the CDRs and FRs are properly positioned. The VH and VL domains and their respective constant regions (CH1 for VH and CL for VL) form the Fab (Fragment antigen-binding) antibody fragment. These regions are joined through the hinge region in the heavy chain, allowing for flexibility. Bioinformatics tools ensure the joined sequences are correctly annotated and in the proper format, with structural modeling tools like PyMOL or Chimera employed to visualize the antibody structure and verify correct domain joining. Functional validation is performed by expressing the recombinant antibody in a suitable system, such as mammalian cells, and conducting antigen-binding assays to confirm functionality.

After accurately recognizing the VH and VL domains that are unique for each antibody and dependent on the entire sequence. The VH and VL are joined using a flexible linker that must be long enough to prevent structural interference in binding. Table 2 shows data from the discovery exercise showing the binding of antibodies with target proteins.

TABLE 2
Enhanced binding of antibodies in conjugation with transferrin protein

| | | | | | | | | | | |
|---|---|---|---|---|---|---|---|---|---|---|
| Protein-protein | AG | Kd (M) | ICs | ICs | ICs | ICs | ICs | ICs | NIS | NIS |
| complex | (kcal | at ° C. | charged- | charged- | charged- | polar- | polar- | apolar- | charged | apolar |
| | mol-1) | | charged | polar | apolar | polar | apolar | apolar | | |
| Aducanumab | | | | | | | | | | |
| Aducanumab-AB | -18.1 | 5.10€-14 | 22 | 20 | 64 | 1 | 19 | 29 | 21.20 | 40.10 |
| Transferrin- | -21.3 | 2.60€-16 | 39 | 27 | 83 | 1 | 18 | 43 | 28.50 | 35.50 |
| (G4S)- | | | | | | | | | | |
| Aducanumab-Aß | | | | | | | | | | |
| Transferrin- | -29.2 | 4.10e-22 | 32 | 33 | 136 | 7 | 38 | 79 | 28.90 | 35.70 |
| (G4S)3- | | | | | | | | | | |
| Aducanumab-Aß | | | | | | | | | | |
| Donanemab | | | | | | | | | | |
| Donanemab-Aß | -13.4 | 1.40€-10 | 8 | 7 | 30 | 2 | 18 | 33 | 21.39 | 37.50 |
| Transferrin- | -15.5 | 4.20€-12 | 8 | 3 | 43 | 1 | 23 | 57 | 28.81 | 35.00 |
| (G4S)- | | | | | | | | | | |
| Donanemab-Aß | | | | | | | | | | |
| Transferrin- | -30.8 | 2.40€-23 | 36 | 12 | 104 | 8 | 58 | 155 | 28.59 | 35.10 |
| (G4S)3- | | | | | | | | | | |
| Donanemab-Aß | | | | | | | | | | |
| Crenezumab | | | | | | | | | | |
| Crenezumab-AB | -13.1 | 2.6e-10 | 12 | 18 | 48 | 4 | 10 | 24 | 20.46 | 40.06 |
| Transferrin- | -20.3 | 1.3€-15 | 27 | 36 | 89 | 5 | 20 | 35 | 27.53 | 36.66 |
| (G4S)- | | | | | | | | | | |
| Crenezumab-AB | | | | | | | | | | |
| Transferrin- | -28.4 | 1.6e-21 | 33 | 54 | 97 | 20 | 63 | 95 | 27.87 | 37.12 |
| (G4S)3- | | | | | | | | | | |
| Crenezumab-AB | | | | | | | | | | |
| Gantenerumab | | | | | | | | | | |
| Gantenerumab- | -13.1 | 2.6e-10 | 12 | 18 | 48 | 4 | 10 | 24 | 20.46 | 40.06 |
| AB | | | | | | | | | | |
| Transferrin- | -25.6 | 1.6e-19 | 43 | 31 | 99 | 5 | 32 | 51 | 28.26 | 35.75 |
| (G4S)- | | | | | | | | | | |
| Gantenerumab- | | | | | | | | | | |
| AB | | | | | | | | | | |
| Transferrin- | -19.1 | 9.7e-15 | 26 | 29 | 74 | 4 | 21 | 35 | 27.99 | 36.48 |
| (G4S)3- | | | | | | | | | | |
| Gantenerumab- | | | | | | | | | | |
| AB | | | | | | | | | | |
| Solanezumab | | | | | | | | | | |
| Solanezumab-AB | -25.6 | 1.6e-19 | 34 | 20 | 101 | 6 | 36 | 58 | 19.60 | 42.33 |
| Transferrin- | -23.6 | 4.8€-18 | 15 | 35 | 53 | 23 | 71 | 68 | 28.14 | 36.17 |
| (G4S)- | | | | | | | | | | |
| Solanezumab-AB | | | | | | | | | | |
| Transferrin- | -27.8 | 4e-21 | 39 | 25 | 105 | 12 | 47 | 40 | 27.97 | 36.25 |
| (G4S)3- | | | | | | | | | | |
| Solanezumab-AB | | | | | | | | | | |

ΔG (kcal mol-1) is the predicted value of the binding affinity, expressed in kilocalories per mole, indicating the energy change associated with forming a complex between molecules. It represents the difference in energy between the bound and unbound states of a ligand and its target, indicating the spontaneity of the binding process. A more negative ΔG signifies stronger binding affinity, meaning the interaction is thermodynamically favorable, and the ligand binds tightly and specifically to the target. Conversely, a less negative or positive ΔG suggests weaker binding affinity and less favorable interaction, indicating the ligand may dissociate more readily. A negative ΔG also indicates spontaneous binding, essential for effective ligand-target interactions in biological systems. At the same time, a positive ΔG denotes non-spontaneous binding, which typically requires energy input and is undesirable in drug design. Additionally, a more negative ΔG implies a more stable ligand-target complex, crucial for drug efficacy as it ensures prolonged interaction with the target. In contrast, a less negative or positive ΔG suggests an unstable complex prone to dissociation. Free energy calculations predict the likelihood of successful binding, aiding drug discovery and development by allowing researchers to prioritize compounds with favorable binding properties. By understanding free energy changes, researchers can optimize ligands to enhance binding affinity and stability, leading to more effective drugs. Free energy also comprises enthalpic and entropic contributions, helping to dissect the driving forces behind binding, such as hydrogen bonds and van der Waals interactions for enthalpy and the release of water molecules for entropy. It is the most critical indicator of the strength of binding.

Kd (M or molarity indicating the molar concentration of the ligand) at the default 25° C.″, representing the equilibrium concentration of dissociated molecules in solution. Mathematically, it is given by the ratio of the rate constants for dissociation (koff) and association (kon): Kd=koff/kon. A higher Kd value indicates that a higher ligand concentration is required to occupy half of the binding sites. This suggests a weaker binding affinity between the ligand and the target. In practical terms, the ligand dissociates more readily from the target, implying faster dissociation. Thus, a ligand with a lower Kd is generally preferred, indicating a more robust and stable interaction with the target.

Intermolecular Contacts (ICs) are the specific points of interaction between the ligand and the target molecule within a defined distance threshold (5.5 Å in this case). Generally, it indicates a more robust and extensive interaction between the ligand and the target. Lower number of ICs: This may indicate weaker binding affinity and less stable interactions. More contacts can mean a more stable and specific binding. Charged-Charged Interactions between oppositely charged groups (ionic interactions) and Charged-Polar Interactions between charged and polar (partially charged) groups are typically more favorable as they involve strong ionic and hydrogen bonding interactions, respectively—charged-Apolar Interactions between charged groups and nonpolar groups (less common, often less favorable).

Polar-polar interactions occur between hydrophilic groups that can form hydrogen bonds and dipole-dipole interactions. These interactions are significant because hydrogen bonds are strong and directional, contributing to the stability and specificity of the ligand-protein complex. Dipole-dipole interactions enhance binding affinity by aligning dipoles in a favorable orientation. Additionally, polar-polar interactions improve solubility in aqueous environments, which is crucial for biological activity and drug delivery, and help form hydration shells around the binding site, influencing binding dynamics and stability. Higher scores in polar-polar interactions generally indicate stronger, more specific binding due to increased hydrogen bonding and dipole interactions. Lower scores suggest weaker interactions and potentially less stable binding.

Polar-apolar interactions occur between hydrophilic and hydrophobic groups. These interactions help position the ligand correctly within the binding site, potentially causing conformational changes that facilitate stronger interactions. While weaker than other interactions, they are transitional in the initial binding stages. Polar-apolar interactions also influence the ligand's solubility and bioavailability by balancing polar regions that enhance solubility with apolar regions that improve membrane permeability. Overall, they contribute to the binding kinetics by balancing the forces involved in the binding process. Higher scores in polar-apolar interactions indicate better positioning and initial ligand recognition. In comparison, lower scores suggest fewer effective interactions that may not optimally guide the ligand into the binding site.

Polar-polar interactions occur between hydrophilic groups that can form hydrogen bonds and dipole-dipole interactions. These interactions are significant because hydrogen bonds are strong and directional, contributing to the stability and specificity of the ligand-protein complex. Dipole-dipole interactions enhance binding affinity by aligning dipoles in a favorable orientation. Additionally, polar-polar interactions improve solubility in aqueous environments, which is crucial for biological activity and drug delivery, and help form hydration shells around the binding site, influencing binding dynamics and stability. Higher scores in polar-polar interactions generally indicate stronger, more specific binding due to increased hydrogen bonding and dipole interactions. Lower scores suggest weaker interactions and potentially less stable binding.

A higher number of ICs is generally better, indicating more robust and stable interactions. Favorable contacts (charged-charged, polar-polar, and apolar-apolar) are significant.

Non-interacting surface (NIS %) refers to the proportion of the ligand or target surface that does not participate in intermolecular interactions. NIS Charged: The percentage of the charged surface area not involved in interactions. It Indicates that a more significant portion of the charged surface is not involved in binding, which might suggest suboptimal electrostatic complementarity. Lower NIS Charged is better, indicating that the charged regions effectively participate in interactions, contributing to binding affinity. Higher NIS Apolar demonstrates that a more significant portion of the apolar surface is not involved in binding. This might suggest that the hydrophobic interactions need to be maximized. Lower NIS Apolar indicates better utilization of apolar regions for binding, which is favorable for hydrophobic interactions. It is better, as it demonstrates that the apolar regions effectively participate in hydrophobic interactions, contributing to binding stability.

In a binding study, a higher number of intermolecular contacts (ICs), particularly those involving favorable interactions (charged-charged, polar-polar, apolar-apolar), is generally desirable. Lower NIS charged and NIS apolar values are preferred, as they indicate better binding utilization of the molecular surfaces. This combination correlates with more robust, stable, and specific ligand-target interactions.

Based on the above exercise, we chose (G4S)3 as our non-cleavable linkage to form the scFvs.

The scFvs that act inside cells must enter the cell where the targets reside. While the smaller size of scFv compared to total antibody enhances the crossing across cell boundary, a substantially higher transit of scFvs can be achieved if they are allowed to piggyback on a transcytosis medium. One is the transferrin protein, a glycoprotein that binds and transports iron into cells via the transferrin receptor, which is highly expressed on the surface of many cells, including cancer cells. Transferrin-bound scFvs can utilize the transferrin receptor-mediated endocytosis pathway, increasing the uptake of the antibody into cells, particularly cancer cells that often overexpress transferrin receptors. This approach could improve the selectivity of the antibody, reducing off-target effects and lowering the required dosage. However, transferrin ((P02787)) is 698 AA long and adds substantial weight to scFv. Since the binding to the transferrin receptor is primarily triggered by its N-methyl lobe, which has substantially lower molecular weight, it was chosen instead to induce transcytosis. The N-terminal lobe of the Serotransferrin Chain A transferrin (1a8e) (22-350 gene PRO1400, TF) has 328 amino acids that are just as efficient in creating the binding with much lower molecular weight.

Additionally, the N-methyl transferrin lobe binding can improve the pharmacokinetics of the scFv, leading to a longer circulation time in the bloodstream and better distribution to the tumor site or across the blood-brain or blood-eye barrier. It is worth noting that the N-methyl lobe of transferrin can help deliver both the scFv and iron, potentially enhancing the metabolic activity of rapidly proliferating cancer cells and making them more susceptible to scFv-mediated cytotoxicity. The endocytic pathway can also help the scFv escape endosomes and reach its intracellular target, potentially overcoming resistance mechanisms related to scFv uptake and degradation.

The efficacy of the scFV-N-methyl transferrin lobe can be substantially increased by blocking the conjugate's exocytosis by letting the linkers connecting the scFv and the N-methyl transferrin lobe break in a specific cellular environment, thereby releasing the scFv that will stay inside the cell for a longer time.

The peptide linkers were chosen to enable recombinant or mRNA production of the scFv conjugate. Several linkers have been identified that respond to enzymatic and pH changes. For example, a linker sensitive to Cathepsin B and acidic pH can ensure specific release in the brain's environment.

A major class of environment-sensitive linkers is that of MMP (matrix metalloproteinase)-sensitive. Specific examples of MMP-sensitive linkers that react to the presence of matrix metalloproteinases are Pro-Leu-Gly-Leu-Trp-Ala, which is Cleaved by MMP-2 and MMP-9; Gly-Pro-Leu-Gly-Ile-Ala-Gly-Gln, which is Cleaved by MMP-9; and Arg-Pro-Leu-Ala, which is Cleaved by MMP-2 and MMP-9. Another standard linker is GPLGVRG.

Redox-sensitive linkers are essential in designing brain applications because they respond to the reductive environment within cells. One widely used redox-sensitive linker is the disulfide bond (Cys-Cys), which can be cleaved by reducing agents such as glutathione. Glutathione is abundant in the intracellular environment, especially within the cytosol of brain cells. It is a tripeptide composed of glutamine, cysteine, and glycine, playing a crucial role in maintaining the redox balance and protecting neurons from oxidative stress. The high glutathione concentration in the brain ensures that disulfide bonds can be efficiently cleaved. Another redox-sensitive linker is CPC.

Cathepsin-sensitive linkers are peptide sequences designed to be cleaved by specific cathepsins. They facilitate the controlled release of therapeutic agents in environments where these enzymes are active. Examples include Gly-Phe-Leu-Gly Ala-Leu-Ala-Leu.

Designing a sequence that includes cleavable linkers requires adding protective groups to ensure targeted cleavage within specific cells, such as cancer cells. The core cleavable sequence, such as Val-Cit (Valine-Citrulline), is chosen for its susceptibility to specific enzymes like Cathepsin B, which is prevalent in cancer cells. To ensure the linker remains inactive until it reaches the target environment, a protective group, such as Phe-Lys (Phenylalanine-Lysine), can be employed. This group masks the cleavable sequence, preventing premature cleavage. The protective group is stable in extracellular environments and brain tissue but is designed to be removed by intracellular proteases that are more active in target cells. Once inside the target cell, the protective group is cleaved, exposing the Val-Cit sequence, which can then be cleaved explicitly by Cathepsin B, releasing the therapeutic payload. This design strategy ensures that the linker remains intact during circulation and in non-target tissues, only breaking down within the target cells, thus providing targeted delivery and minimizing off-target effects.

A different cleavable linker and protector is needed if targeting non-cancerous cells. The adaptability of the selection process is evident in the design of a sequence tailored to the specific enzymes and conditions prevalent in the target non-cancerous cells. For instance, if targeting lysosomal enzymes in non-cancerous cells, a better choice is GFLG (Gly-Phe-Leu-Gly), which is sensitive to Cathepsin D, a lysosomal protease typical in many cell types. A protective group like Phe-Lys (Phenylalanine-Lysine) can mask the cleavable sequence, ensuring stability outside the target cells. This protective group would be stable in extracellular environments and the brain but cleaved by intracellular proteases once inside the non-cancerous cells. After these proteases remove the protective group, the GFLG sequence is exposed and can be specifically cleaved by Cathepsin D, allowing the release of the therapeutic agent. This tailored approach ensures that the linker remains intact in non-target tissues and only breaks down within the designated non-cancerous cells, providing targeted delivery and reducing off-target effects.

The additional GSG spacers on either side of the linker's domains ensure the overall construct maintains flexibility and proper spatial arrangement for efficient cleavage. This design provides stability at neutral pH, keeping the linker intact during circulation and crossing barriers like the blood-brain or blood-eye barriers. The slightly acidic pH inside lysosomes or in specific cellular microenvironments can facilitate the removal of the HH protective group, exposing the GFLG sequence for subsequent cleavage. Combining these elements ensures targeted delivery and controlled release within specific cells in the brain or eye, making it suitable for crossing biological barriers and ensuring efficient therapeutic delivery in the target tissue.

If the intent is to cross the blood-brain barrier (BBB) or the blood-eye barrier (BEB), a different cleavable linker and protective group would be required, tailored to the unique environment of these barriers and the target cells within the brain or eye. The brain's extracellular environment is slightly more acidic than blood. Linkers sensitive to acidic pH (around pH 6.5-6.8) can be designed to break specifically in the brain. Examples include the GFLG, which is sensitive to Cathepsin D, a lysosomal protease that could be used to cleave in mildly acidic conditions. Another pH-sensitive linker is DPG.

Like Phe-Lys (Phenylalanine-Lysine), the protective group would mask the cleavable sequence, ensuring it remains inactive during circulation and crossing the BBB or BEB. This protective group would be stable at the neutral pH of the blood and the brain or eye extracellular space but would be cleaved by intracellular proteases once inside the target cells. The optimal cleavable brain or eye entry linker is GFLG, protected by HH and GSG as spacers on both sides of GFLG: GSG-HH-GFLG-GSG; this is a good choice for targeting specific cells in the brain or eye, considering the intended use and enzymatic environment. The GSG spacer (Gly-Ser-Gly) helps to provide flexibility and distance between the functional domains, ensuring proper exposure and accessibility of the cleavable sequence to the target enzymes. The protective group, HH (His-His), can serve as a protective group due to the properties of histidine residues. Histidine residues provide stability at neutral pH but become positively charged in slightly acidic environments (pH ˜6), potentially aiding in removing or exposing the cleavable sequence within the target cell's lysosomal or slightly acidic microenvironments.

After these intracellular proteases are removed, the protective group and the GFLG sequence are exposed. Cathepsin D, present in the lysosomes of the target brain or eye cells, can specifically cleave them. This design ensures that the linker remains intact while crossing the BBB or BEB and only breaks down within the target cells in the brain or eye, thus providing targeted delivery and minimizing off-target effects.

The scFvs have the additional advantage of expression in bacteria, such as Escherichia coli, due to the simplicity, cost-effectiveness, and high yield of bacterial expression systems. Additionally, scFvs can be encoded easily as mRNA and delivered directly into cells, removing post-translational modification limitations when expressing using bacteria and ensuring the correct modifications are needed for stability and function. In eukaryotic cells, protein folding mechanisms and chaperones are in place to ensure proper folding of complex proteins. This increases the likelihood that a scFv will fold correctly and maintain its functional conformation. Eukaryotic cells, particularly the endoplasmic reticulum, provide an environment conducive to forming disulfide bonds, which are critical for the structural integrity of many antibodies. Eukaryotic cells have mechanisms to assist in the proper folding and solubility of proteins, reducing the risk of aggregation often seen in bacterial systems. When the scFv is produced directly inside the target cells, this approach bypasses the need for purification and refolding processes associated with bacterial expression systems, potentially leading to a more active and functional protein.

### DETAILED DESCRIPTION OF THE INVENTION

Binding the N-methyl lobe of transferrin to a single-chain variable fragment (scFv) involves deciding whether to attach it to the variable heavy (VH) or variable light (VL) chain. Attaching transferrin to either the VH or VL chain could affect the folding and stability of the scFv. The linker between VH and VL is designed to allow proper folding and antigen binding.

When engineering an scFv, the C-terminus is a common site for attaching additional functional domains, such as the N-terminal lobe, tags, or other proteins, because it is typically more accessible and does not interfere with the antigen-binding region: N-terminus - - - scFv (VH-VL) - - - Linker - - - N-methyl Transferrin lobe - - - C-terminus. The scFv (comprising VH and VL regions linked by a peptide linker) retains its antigen-binding capability in this configuration. A flexible linker connects the C-terminus of the scFv to the transferrin, ensuring both domains fold correctly and function independently.

Using multiple types of cleavage linkers that respond to different environmental triggers can increase the specificity and efficiency of cleavage, especially in complex environments like the brain or tumor microenvironments. This strategy can provide a more controlled and targeted release of therapeutic agents by ensuring that the cleavage occurs only in the presence of the desired conditions.

An example of a single cleavage linker at the C-terminus might be sufficient for specific applications: N-terminus - - - N-methyl Transferrin lobe - - - (Flexible Linker) - - - (MMP-Sensitive Linker) - - - scFv - - - VH - - - (Flexible Linker) - - - scFv - - - VL - - - C-terminus.

An example configuration with multiple cleavage linkers: N-terminus - - - scFv VH - - - (Flexible Linker)-scFv VL - - - (Flexible Linker) - - - MMP-Sensitive Linker) - - - (Flexible Linker) - - - (pH-Sensitive Linker) - - - (Flexible Linker) - - - (Redox-Sensitive Linker) - - - (Flexible Linker) - - - N-methyl Transferrin lobe - - - C-terminus.

The ideal flexible linker is (G4S)3.

1. A fusion protein comprising a single chain variable fragment (scFv) of eculizumab antibody, conjugated with an N-terminal lobe of transferrin protein (PDB: 1a8e; 22-350 gene PRO1400, TF) using an environment-sensitive cleavable linker or linkers.

2. The fusion protein of claim 1, wherein the scFv comprises a variable heavy chain (VH) and variable light chain (VL) conjugated using a (G4S)3 linker.

3. The fusion protein of claim 1, wherein the N-terminal lobe of transferrin is fused to either the carboxyl-terminus or the amino-terminus of the scFv, either monovalently or multivalently.

4. The fusion protein of claim 1, wherein the cleavable linker is sensitive to pH, redox potential, Cathepsin B or D, or MMP (matrix metalloproteinase)-sensitive, or a combination thereof.

5. The fusion protein of claim 4, wherein the cleavable linker is GSG-HH-GFLG-GSG GFLG.

6. The fusion protein of claim 1, wherein the fusion protein is produced using recombinant expression in bacteria or mammalian cells or delivered to the body by encoding through an mRNA composition.
